(19) United States
(12) Patent Application Publication
(10) Pub. No.: US 2015/0118664 A1
(43) Pub. Date: Apr. 30, 2015

(54) HUMAN MEMORY CHUNK CAPACITY TEST

(71) Applicant: Eugen Tarnow, Fair Lawn, NJ (US)

(72) Inventor: Eugen Tarnow, Fair Lawn, NJ (US)

(73) Assignee: Eugen Tarnow, Fair Lawn, NJ (US)

(21) Appl. No.: 14/066,195

(22) Filed: Oct. 29, 2013

(51) Int. Cl. A61B 5/16

(52) U.S. Cl. CPC A61B 5/16
USPC 434/236

(57) ABSTRACT

Several methods are used to better define and optimize item sets for short-term memory tests with respect to chunkiness and homogeneity, including dynamically changing the test item set to make it have as few chunkable items as possible. A resulting item set allows the test taker to see in an intuitive way how her or his memory has a limited capacity of, typically, three non-chunkable items. The test can be done, inter alia, via computer, via the telephone, and via robocalling. It can, inter alia, provide mass screening for Alzheimer's disease and detect early Alzheimer's disease in drug trials.

### COPYRIGHT NOTICE

A portion of the disclosure of this patent document contains material that is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### CROSS-REFERENCE TO RELATED APPLICATIONS

This application is related to my copending application, Ser. No. 13/782,011, filed Mar. 1, 2013, which shows a system and method that tests for presence of neurological change using an aspect of free recall called the recency-primacy shift.

### SEQUENCE LISTING OR A COMPUTER PROGRAM LISTING

A listing of a computer program that implements the method of the present application is provided before the Claims.

### PRIOR ART

The following lists some prior art that presently appears relevant.

### U.S. Patents

| Patent or | Kind | Issue or Pub. | |
|---|---|---|---|
| Pub. Nr. | Code | Date | Patentee or Applicant |
| 4,770,636 | B1 | 1988 Sep. 13 | Herman Buschke |
| 5,230,629 | B1 | 1993 Jul. 27 | Herman Buschke |
| 6,306,086 | B1 | 2001 Oct. 23 | Herman Buschke |
| 6,485,417 | B1 | 2002 Nov. 26 | Henry M. Bowles, Theodore |
| | | | D. Langley |
| 6,689,058 | B2 | 2004 Feb. 10 | Herman Buschke |
| 6,964,638 | B2 | 2005 Nov. 15 | Alexis Theodoracopulos, et al. |
| 7,070,563 | B2 | 2006 Jul. 04 | Herman Buschke |
| 7,314,444 | B2 | 2008 Jan. 01 | Herman Buschke |
| 7,942,828 | B2 | 2011 May 17 | Steven B. Lowen et al. |

### U.S. Published Patent Applications

| Patent or Pub. Nr. | Kind Code | Publication Date | Patentee or Applicant |
|---|---|---|---|
| 2005/0196735 | A1 | 2005-09- 08 | Herman Buschke |
| 2011/0236864 | A1 | 2011-09-29 | John Wesson Ashford |
| 2008/0057483 | A1 | 2008-03-06 | Lawrence H Avidan |
| 2008/0070207 | A1 | 2008-03-20 | Lawrence H Avidan |

### Non-Patent Literature Documents in Alphabetical Order by Author

ANDERSON, D. E., VOGEL, E. K., & AWH, E. (2011). Precision in visual working memory reaches a stable plateau when individual item limits are exceeded. The Journal of neuroscience, 31(3), 1128-1138.
ANDERSON, D. E., VOGEL, E. K., & AWH, E. (2013). A common discrete resource for visual working memory and visual search. Psychological science, 24(6), 929-938.
AWH, E., BARTON, B., & VOGEL, E. K. (2007). Visual working memory represents a fixed number of items regardless of complexity. Psychological science, 18(7), 622
BROADBENT, D. E. (1975) The Magic Number Seven After Fifteen Years, in Studies in Long Term Memory, A. Kennedy & A. Wilkes (Eds.), John Wiley & Sons
BUNTING, M., COWAN, N., & SCOTT SAULTS, J. (2006). How does running memory span work? The Quarterly Journal of Experimental Psychology, 59(10), 1691-1700.
CARPENTER S K (2005). Some neglected contributions of Wilhelm Wundt to the psychology of memory. Psychol Rep. 2005 August; 97(1):63-73.
CHEN, Z., & COWAN, N. (2005). Chunk limits and length limits in immediate recall: A reconciliation. Journal of experimental psychology. Learning, memory, and cognition, 31(6), 1235.
COWAN, N. (2001). The magical number 4 in short-term memory: A reconsideration of mental storage capacity. Behavioral and brain sciences, 24(1), 87-114.
COWAN, N., JOHNSON, T. D., & SCOTT SAULTS, J. (2003). Capacity limits in list item recognition: Evidence from proactive interference. Memory, 13(3-4), 293-299.
COWAN, N. (2004). Working memory capacity. Psychology Press.
CUSACK, R., LEHMANN, M., VELDSMAN, M., & MITCHELL, D. J. (2009). Encoding strategy and not visual working memory capacity correlates with intelligence. Psychonomic bulletin & review, 16(4), 641-647.
DEMPSTER, F. N. (1981). Memory span: Sources of individual and developmental differences. Psychological Bulletin, 89(1), 63-100.
FRIENDLY, M., FRANKLIN, P. E., HOFFMAN, D., & RUBIN, D. C. (1982). The Toronto Word Pool: Norms for imagery, concreteness, orthographic variables, and grammatical usage for 1,080 words. Behavior Research Methods & Instrumentation, 14(4), 375-399.
GLANZER, M., & RAZEL, M. (1974). The size of the unit in short-term storage. Journal of Verbal Learning and Verbal Behavior, 13(1), 114-131.
GOBET, F., LANE, P. C., CROKER, S., CHENG, P. C., JONES, G., OLIVER, I., & PINE, J. M. (2001). Chunking mechanisms in human learning. Trends in cognitive sciences, 5(6), 236-243.
GRAESSER, A., & MANDLER, G. (1978). Limited processing capacity constrains the storage of unrelated sets of words and retrieval from natural categories. Journal of Experimental Psychology: Human Learning and Memory, 4(1), 86-100.
HALFORD, G. S., COWAN, N., & ANDREWS, G. (2007) Separating cognitive capacity from knowledge: a new hypothesis. TRENDS in Cognitive Sciences, 11(6), 237.
HESSELS, M. G. (2002). Performance of Adolescents with Moderate Mental Retardation on a Working Memory Task and Analysis of their Response Patterns. Journal of Cognitive Education and Psychology, 2(3), 283-300.
LAMING D (2008), An improved algorithm for predicting free recalls, Cognitive Psychology 57 179-219
MCELREE, B. (1998). Attended and non-attended states in working memory: Accessing categorized structures. Journal of Memory and Language, 38(2), 225-252.
MCELREE, B. (2001). Working memory and focal attention. Journal of experimental psychology. Learning, memory, and cognition, 27(3), 817.
MILLER, G. A. (1956) The magical number seven, plus or minus two: Some limits on our capacity for processing information. Psychological Review 63:81-97.
MULLIGAN, N. W. (1997). Attention and implicit memory tests: The effects of varying attentional load on conceptual priming. Memory & Cognition, 25(1), 11-17.
MURDOCK B B (1962). The serial position effect of free recall. Journal of Experimental Psychology 64(5) 482-488.
MURDOCK, B., & METCALFE, J. (1978). Controlled rehearsal in single-trial free recall. Journal of Verbal Learning and Verbal Behavior, 17(3), 309-324.
OBERAUER, K. (2002). Access to information in working memory: Exploring the focus of attention. Journal of Experimental Psychology-Learning Memory and Cognition, 28(3), 411-421.
OBERAUER, K., & BIALKOVA, S. (2009). Accessing information in working memory: Can the focus of attention grasp two elements at the same time?. Journal of Experimental Psychology: General, 138(1), 64.
OBERAUER, K., & HEIN, L. (2012). Attention to information in working memory. Current Directions in Psychological Science, 21(3), 164-169.
ROUDER, J. N., MOREY, R. D., COWAN, N., ZWILLING, C. E., MOREY, C. C., & PRATTE, M. S. (2008). An assessment of fixed-capacity models of visual working memory. PNAS, 105(16), 5975-5979.
ROBNAGEL, C. S. (2004). Lost in thought: Cognitive load and the processing of addressees' feedback in verbal communication. Experimental Psychology (formerly Zeitschrift für Experimentelle Psychologie), 51(3), 191-200.
SIMON, H. A. (1974). How big is a chunk. Science, 183(4124), 482-488.
SPERLING, G. (1960). The information available in brief visual presentations. Psychological monographs: General and applied, 74(11), 1-29.
TARNOW, E. (2010). Why The Atkinson-Shiffrin Model Was Wrong From The Beginning WebmedCentral NEUROLOGY 2010; 1(10):WMC001021
TARNOW, E. (2008). Response probability and response time: a straight line, the Tagging/Retagging interpretation of short-term memory, an operational definition of meaningfulness and short-term memory time decay and search time. COGNITIVE NEURODYNAMICS, 2(4), 347-353.
TATUM, B. C., NEBEKER, D. M., & SORENSEN, S. W. (1991). Work Standards, Productivity, and Quality (No. NPRDC-TR-91-12). NAVY PERSONNEL RESEARCH AND DEVELOPMENT CENTER SAN DIEGO CA.
YANTIS, S. (1992). Multielement visual tracking: Attention and perceptual organization. Cognitive psychology, 24(3), 295-340.

### What is a “Chunk” and how Many Chunks can we Remember?

The above references, discussed infra, relate to short-term memory of humans, which is an important part of our everyday life. Variations and deficiencies in its properties are important for identifying gifted individuals, diagnosing memory disorders, and probing temporary or permanent attention issues. One of the more sought-after properties of short-term memory is its storage capacity.

In the end of the 1800s, Wundt (see Carpenter, 2005, supra) found, from varying metronome-beat experiments, that the “scope of consciousness,” as far as recognition is concerned, is limited to 18 different metronome beats. (Sequences of “tic-tocks” were varied according to the time interval between beats and the number of beats was increased until the participant could no longer determine whether the two sequences were the same or different).

George Miller (1956) noted, “everybody knows that there is a finite span of immediate memory and that for a lot of different kinds of test materials this span is about seven items in length.” He coined the “chunk” as the “unit” of what is stored in memory as opposed to counting the apparent bits information content (for example, the number of different metronome beats, the number of characters, phonemes, or the total pronunciation times of words). Thus “202” is one chunk for those who are familiar with the area code of Washington D.C. but perhaps two or three chunks for those who are not. IBM is a chunk for those familiar with the company but perhaps two or three chunks for those who are not.

Sperling (1960) showed that the number of items that can be visually remembered starts out at twelve but decreases quickly to four as the picture on the retina disappears.

Murdock (1962) showed that when presented with a list of 10-40 words, subjects remember an average of only six to nine words.

Simon (1974) argues that the capacity of short-term memory is five, not seven chunks.

Cowan (2001, for a review see Cowan, 2004) claims the true capacity of short-term memory is not seven but four chunks.

Glanzer & Razel (1974) argue that the capacity is just two chunks. A review by Oberauer & Hein (2012) of reaction time experiments, claims that although short-term memory probably has a capacity limit of four chunks, there is only at most a single item in the center of attention. If anything else is added a subject will usually associate it with the first item to create a larger item, which still is only one item (McElree, 1998; Oberauer et al, 2009).

Determining which limit on short term memory is correct—one, two, four, seven or more—is important for understanding and characterizing short-term memory and is, as we have seen, not easy to answer. It is further complicated by the fact that the definition of the unit of measurement is an issue.

Simon (1974) refers to the term “chunk” as an “artfully vague” term for “a particular amount that has specific psychological significance” and but states, “the capacity of short-term memory, measured in chunks, is independent of the material of the chunks—five chunks of words, five chunks of digits, five chunks of colors, five chunks of shapes, five chunks of poetry or prose.” Thus, Simon equates “psychological significance” with a general five-chunk capacity of short-term memory.

Halford et al (2007) enumerates a number of experiments that shows that two to six chunks is the limit of short-term memory. These experiments involved a multitude of probes: spatial arrays (Luck & Vogel, 1997), perfect recall of a series (Broadbent, 1975), running span (a presented list ends unpredictably and the subject is asked to recall the last few items; Bunting et al, 2006), a span with distraction or suppression (Cowan, 2004), categorical retrieval (Graesser & Mandler, 1978), memory updating (Oberauer, 2002), probed recall (in which a particular item is asked to be recalled; McElree, 2001 and Cowan et al, 2003), multiobject tracking (Yantis, 1992), reconstruction by chunks (Gobet et al, 2001), and recollection by chunks (Tulving & Patkau, 1962 and Chen & Cowan, 2005). Halford et al (2007) preface several of these experimental results with “presumably” or “assuming” because the experiments themselves are difficult to understand or require additional non-trivial assumptions. To this list can be added experiments that need to use complex terms or complex mathematical analysis to get a chunk count.

The “precision of recognition” of geometric objects increases up to 3 items after which it plateaus (Anderson et al, 2011).

Awh et al. (2007) showed that visual memory keeps a fixed number of objects but has limited resolving power—if the objects are too similar change detection becomes erroneous.

Rouder et al (2008) showed that a fixed memory capacity (either an item is remembered or it is not) is indicated by what is called the Receiver Operating Characteristics. The curves that plot hit rate versus false alarm rate are straight lines with a unit slope.

Zhang and Luck (2008) studied the balance between capacity and resolution and found that there is no trade-off between the two but rather the capacity to recall is fixed at 3.

There is no simple and intuitive way to show what the number of items that can be remembered. Why should something of “psychological significance” be so difficult to explore? One reason is the lack of a formal way to minimize chunking of an item set, there is no proof that subjects are not “chunking” the items asked to remember; this leaves any experiment vulnerable towards criticism that subjects can associate several items together into one item. Typically item set preparers will just give up if an item set seems to allow chunking.

For example, digits were presumed to always be chunkable, e.g., two digits might be chunked into just one item (Dempster, 1981), and thus integers are not used. Instead researchers focused on visually displaying strange looking symbols to avoid facing the chunkiness of the item set, assuming that the stranger the item set, the less likely it can be chunked. Thus an item set can contain several cut-up circles with the cut-up parts pointing in different directions (Anderson et al., 2013) or other unusual shapes (see http://alertometer.com/shapes/). While this set may look strange and somewhat disconnected from every-day experiences there is nevertheless no proof that it is not chunkable.

Hessels (2002) claimed to minimize chunking of dot patterns on a Mr. Peanut figure by eliminating “symmetrical positions (e.g., two arms), identical positions on consecutive items, and obvious patterns”. However he never defined the chunking concept nor proved that his actions did minimize chunking.

Mulligan (1997) studied “attentional loads” using items that would require different amounts of attention. He constructed these items to minimize chunking by avoiding repetition of letters or digits: “Nonzero loads were constructed by randomly selecting items from a set of digits (1-9) and a set of letters (B, C, D, F, G, H, J, K, L) according to the following rules: (1) digits and letters occupied alternating positions, with a digit in the first position (attentional loads of 1 consisted of a single digit), and (2) no repetition of digits or letters within a load. It was thought that the use of these materials and rules would help to minimize chunking.”

A similar procedure was used by Rossnagel (2004).

Tatum et al (1991) includes a questionnaire with questions about how much chunking the respondents do while typing but this is not directly relevant to a chunking scale of memory test items.

### Issue of Rehearsal Makes Interpretations of Experimental Results Difficult

It is known that rehearsal (a subject repeatedly presenting items to himself or herself) plays some role in maintaining short-term memory. This is typically considered a necessary nuisance (Tarnow, 2010) because it is hard to show that everybody rehearses and for those that rehearse, the rehearsal patterns are complex (Laming 2008).

Murdock & Metcalfe (1978) write that, “a controlled-rehearsal procedure may be useful to study rehearsal processes in free recall”. They asked subjects to rehearse aloud to record individual rehearsal patterns and then tried to impose those patterns when they presented lists of 20 words to the subjects. Other writers also tried to impose and test different rehearsal patterns but the issue of rehearsal is like a rainy cloud always hanging over experimental results and ready to empty itself at any moment.

### Item Sets are not Homogenous

Murdock & Metcalfe (1978) describe a standard item set as follows: “The Toronto word pool, a list of 1040 two-syllable common English words not more than eight letters long with homophones, contractions, archaic words, and proper nouns deleted.” Such a manual scan for homogeneity is good but not perfect. No doubt other categories of words can be excluded to make the set more homogenous and, even within a single word category, there may be words that are special in other ways. For example, included in the word pool is “herald” which may be very easy to remember for those with a subscription to The Boston Herald. Investigations such as Friendly et al (1982) have rated words in the Toronto Word Pool on various properties such as probability of usage, but not in terms of how easy the words are to recall or recognize in an experiment; homogeneity is currently only enforced for properties that are not measured in the experiment at hand. The reason for this is that the Toronto Word Pool is thought of as a way to represent the English language, not as a way to homogenously sample the brain.

The following are some relevant patents relating to memory tests and a discussion of each:

Buschke ('636 and '629) tests the user's memory for immediate and delayed recall of single items consisting of random digits in a phone number pattern with 4, 7 or 10 digits. This test encourages non-homogenous chunking in a phone number pattern (202 can be chunked as “2”, “0” and “2” or “20” and “2” or just “202”) and is not useful to determine the number of similar chunks short-term memory can contain.

Buschke ('086, '058, '444, '563, and '735) tests free recall in conjunction with a recognition test for items not first recalled. He weighs the correct responses to get a single number rating to identify disease and drug effects. The test items are chunkable and cannot be used to directly probe the number of chunks short-term memory can contain.

Bowles et al. ('417) test attention using computer game-like presentations of complex shapes (see http://alertometer.com/shapes/) that move around in which the subject is supposed to tell whether all the shapes are the same or not. It is not a straightforward test of the number of chunks in short-term memory.

Theodoracopulos et al ('638) test for changes over time in cognitive test results but does not directly measure the number of chunks in short-term memory.

Teicher et al. ('828) test for fluctuations in attention by looking at cognitive scores as a function of time but does not directly measure the number of chunks in short-term memory.

Avidan ('483 and '207) tests memory in a variety of ways but no detailed descriptions are made of the test item sets.

Ashford tests short-term memory for the presence of Alzheimer's disease using a variety of pictures. There is no direct test for the number of chunks in short-term memory.

### Alternative Delivery Systems for Memory Testing

Typically, memory testing is performed in a laboratory setting, either individually or in a group (Murdock, 1962) or in a hospital (Teicher & Lowen). Buschke ('636) stressed the need for standardized testing that can be carried out “at home as well as in medical offices, clinics, emergency rooms, hospital wards, psychiatric facilities, or nursing homes” and suggested that a portable device would be useful. With recent technological advances, testing is also taking place over the Internet (Avidan '207 and '483). The current generation of older people, however, is inconvenienced and embarrassed by having to go to a clinical setting for testing and often has difficulty using computers or portable testing devices.

### BACKGROUND

### Advantages

According to one or more aspects, my system has one or more of the following advantages:
1. Memory tests can be performed over the telephone, simplifying access to old test takers and significantly lowering the costs of the test administration.
2. A memory test can be done with few list items to allow the limits of short-term memory to be measured straightforwardly without the need for complex mathematics or complex visual test items.
3. This system allows a layperson to see and “feel” their own short-term memory limit without asking a scientist or a computer to analyze the results.
4. The measured limit on short-term memory can be easily probed for memory deficiencies.
5. The memory test shows how short-term recall memory is divided into three items.
6. A controlled rehearsal process can be constructed and probed for memory deficiencies in terms of the ease with which to locate and internally reactivate test items.
7. The system allows minimizing or maximizing associations between memory test items and increasing the homogeneity of a set of memory test items.
8. The number of items presented to a test taker can be slowly increased until the test taker's memory is overloaded. The test taker's response to the overload can be probed for executive decision deficiencies caused by disease or sleepiness or drug usage.
9. The system allows the construction of a cross-cultural memory test by avoiding language specific word associations and limiting it to integers.
10. The cross-cultural test allows memory deficiencies to be compared and defined across international borders.

Further advantages of various aspects will be apparent from the ensuing description and accompanying drawings.

### SUMMARY

I provide a method for determining and constructing the “chunkiness” and homogeneity of an item set to be used for testing human short-term memory. In one aspect, a computer screen begins to display lists of two double-digit numbers that the subject is then asked to recall. Frequently the numbers are remembered in the order they were displayed. The computer screen then displays lists of three double-digit numbers that the subject is asked to recall. Again, the numbers are typically remembered in the order they were displayed. Then the computer screen displays lists of four double-digit numbers. Few, if any, subjects are able to recall all four items. The method allows a tester to probe how many “chunks” short-term memory can contain and how memory breaks down if there is a chunk too many. Prevention of the memory breakdown necessitates a decision-making process for which alertness and attention is required. This test can be used both for time-separated intra-individual comparisons to measure individual memory, decision-making, alertness, and attention, and inter-individual comparisons by measuring similarity of a subject's test profile to a disease/drugged population profile and deviance from a normal population profile. In addition the methods allow scoring the difference between individual current and past test scores as a more accurate measure of the onset and progress of a neurological disease or effects of neuropharmacological drugs.

### DRAWINGS

FIG. 1 shows a block diagram of a first embodiment of my Human Memory Chunk Capacity Test system.

FIG. 1a shows a slight variation of the first embodiment where the test taker can speak the answers into a microphone and have the computational device(s) 101 recognize the speech.

FIG. 2 shows a block diagram of a second embodiment of my system where the presentation of the items and the test taker responses is entered using an Excel spreadsheet.

FIG. 3 is a graph that shows the total number of double-digit numbers subjects can remember as a function of the number of items displayed when the second embodiment is used.

FIG. 4 is a graph that shows the probability of remembering a double-digit number item versus the order of item presentation in the list (whether the number was displayed 1^{st}, 2^{nd}, 3^{rd} or 4^{th}) when the second embodiment is used.

FIG. 5 shows a block diagram of a third embodiment of my system where the computational device(s) 101 can also set up a telephone call using a database of phone numbers in storage device 102. Which phone numbers are called can be determined by statistical sampling performed by 101.

FIG. 6 shows a block diagram of a fourth embodiment of my system where the computational device(s) 101 are connected to the internet and serves up an internet page on a test taker device.

### DEFINITIONS

Chunk (noun): a group of one or more associated items stored in short-term memory.
Chunk (verb): to group two or more associated items in short-term memory.
Chunkable item set: set of N items that can be divided into M<N chunks.
Chunkiness: a measure of how chunkable an item set is. One can define it several ways, for example: Chunkiness=(N-M)/N or Chunkiness=average number of items remembered.
Downchunk: to lower the chunkiness of an item set
Dynamical set change rule: rule that states if an item X from an item set is presented to a test taker, said item set changes according to the rule before the next item presentation. For example, in case of downchunking, a subset from said item set will be excluded in subsequent presentation in order to prevent the items of that subset to be chunked with the presented item X. The trivial dynamical set change rule is exclude a presented item from further presentations.
Homogenous item set: an item set in which each item is as memorable as any other item
Homogeneity: a measure of how homogenous an item set is. One can define it in several ways, for example: Homogeneity=Average Standard deviation of probability of item being remembered. Thus the lower the standard deviation of the probability of being remembered, the higher is the homogeneity.
Non-Trivial Dynamical set change rules: Dynamical set change rules that do not include the trivial dynamical set change rule.
The Recency-Primacy Shift (RPS) is the slope of the difference between the test and control free recall probabilities of presented item as a function of presentation order multiplied by the number of presented items.
The trivial dynamical set change rule: The trivial dynamical set change rule excludes a presented item from further presentations within the same list.
Upchunk: to increase the chunkiness of an item set

### DETAILED DESCRIPTION

### First Embodiment

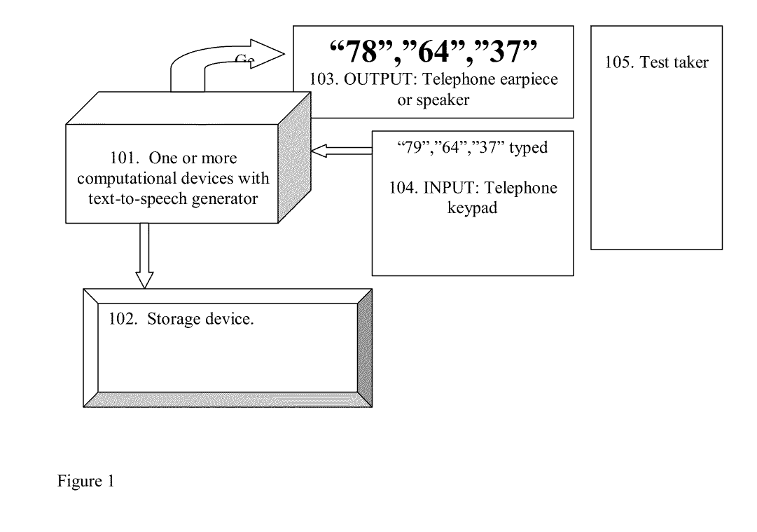

FIG. 1 shows a first embodiment of a system for testing a subject's memory tests over the phone. One or more computational devices 101 presents test items via a telephone earpiece 103. A test taker 105 recalls the test items and enters them into the telephone via a telephone keypad 104. The computational device is connected to a storage device 102 that stores the test information or sends the test information elsewhere via a network.

A slight variation of the embodiment is shown in FIG. 1a in which test taker 105 speaks the recalled items into the telephone microphone 106 and the speech is turned back into numbers via speech recognition software in the computational devices 101. In this embodiment a request can be made by 101 to the test taker to select a language preference from a list. The test taker can use the buttons on the phone to choose from the list (FIG. 1). The test taker can also be requested to name the language into the microphone and have the speech recognition software interpret the response (FIG. 1a).

The system can be used to test the short-term memory of a subject or test-taker in six steps.

First, an item list is prepared and the “chunkiness” (defined above) and “homogeneity” (define above) are measured, after which the chunkiness is minimized and homogeneity is maximized. This allows us to accurately count the number of items a test taker remembers; if the item set used can be chunked, or if some items are much more easily remembered than others, that count is not well defined. We set the chunkiness scale to be the average number of items remembered for a configuration of item set, subject population, and memory test. For example, suppose integers 11-99 are used as the item set. (Items other than integers can be used.)

We measure the initial chunkiness of this item set using the first embodiment by presenting a subset of items from the item set (where the subset includes at least one item and at most all items of the item set) and requesting the test taker to recall the items. The items can be selected in order that they appear in the first item set or be selected at random taking care that no item is selected more than once. Using all of the items (not necessarily all items on each test taker) and many test takers gives us the chunkiness as well as the recall probabilities of each item in the item set.

We then attempt to construct a second item with lower chunkiness set by starting with the first item set but exclude integers that are next to each other: e.g., if 41 is in the item set, 40 and 42 will not be present. This downchunking can be done before the item set is presented, or even better, using a dynamical set change rule (defined above). A dynamical set change rule can shrink the item set after each item is presented. Thus the downchunking is performed at the same time the test is given. For example, if the original item set consists of all two digit numbers and the single item 34 is presented, then the subset {33, 35} is removed from the original item set if the dynamical set change rule is “exclude integers that are next to integers already presented”. This is preferable than to do it before the test is given. In the latter case many more items would have to be excluded (we could exclude every 2^{nd} integer in this case instead of just exclude the two integers next to the presented integer); the subject may more easily recognize patterns in the presented items and get better at guessing what was presented if they no longer remember what was presented. We can further lower the chunkiness of the second item set by removing transpositions. E.g., if 34 is in the second set, 43 will not be present in the third set. If the total memory score is smaller for the second item set in combination with the new dynamical set change rules, than for the first item set, the second item set and the corresponding dynamical set change rules is the preferred combination of item set and dynamical set change rules.

Thus we have systematically improved (lowered) the item set chunkiness by guessing which items can be chunked, removing them and then measuring the new chunkiness. This procedure can also be completely automatic if the computational device randomly substitutes, adds or deletes items in what we can call the first item set to construct a second item set; then compares the chunkiness of the first and second item sets and then renames the item set with the lower chunkiness the next “first item set”; and then goes back to substitute, add or delete items to make a next “second item set” and so on. Importantly, my method quantifies the chunkiness of the item set so that it can be compared with other item sets. This quantification also potentially allows an item set to be improved by further lowering its chunkiness.

To increase the homogeneity of the initial item list, items that are remembered statistically better than other items, according to the test results, are removed. E.g., the two-digit number 37 is probably going to be more easily remembered than other two-digit numbers if the test takers are members of a labor union called “DC-37”. This number would be removed when testing that population to increase the homogeneity of the item list. Integers that are multiples of 10 (20, 30, etc) are easier to remember than the other two-digit integers. So are integers that are multiples of 11 (22, 33 etc), as are the integers 11-20, which are used much more frequently than the other integers. If the probability of remembering the various test items becomes more similar in a second item set than in a first, the probabilities having a smaller standard deviation, the second item set is the more optimal item set and can be used to further increase the homogeneity. Thus we have systematically improved (increased) the item set homogeneity by removing items that are remembered better than other items. This procedure can also be completely automatic if the computational device randomly substitutes, adds or deletes items in what we can call the first item set to construct a second item set; then compares the homogeneity of the first and second item sets and then renames the item set with the highest homogeneity the next “first item set”; and then goes back to substitute, add or delete items to make a next “second item set” and so on. Importantly, my method quantifies the homogeneity of the item set so that it can be compared with other item sets. This quantification also potentially allows an item set to be improved by further increasing its homogeneity.

Once the test item set has been constructed on one set of test takers, we can use the test item set on other test takers.

Second, the test taker acquires an identity to be used to pay for the memory test. This identity can be acquired in many ways: via a credit card number, by buying a specific identity number in a store, via the test taker's phone number, or via a medical, psychological or research establishment that confers an identity number on the test taker, etc. If the subject is required to enter a number into the telephone, this process serves as practice for the subject to enter numbers into the telephone and makes the subsequent input steps more reliable.

Third, the test taker initiates a call (or a researcher using the test taker as a subject, or a medical or psychological provider caring for the test taker, initiates the call or forwards an active call from or to the test taker) to a particular phone number answered by computational device 101 (FIGS. 1 and 1a). The comp charges the caller for the test by either by requesting caller credit card information, requesting a special code previously paid for, by charging the calling phone number or, in the case of a call forwarded from a medical establishment, by charging the medical establishment. The computational device then requests a language preference. The test taker responds via naming the language by a code input into the key pad 104 (FIG. 1) or via a verbal request into the telephone microphone 106 (FIG. 1a).

Fourth, a computer program running on device 101, presents items from the test set using a random item selector, performs dynamical set change, and converts the items to voice in the appropriate language. It presents successive lists of two items (six lists), three items (six lists), and four items (six lists). After each list presentation the list is stored by the computational device on storage device 102.

FIGS. 1 and 1a show how test items 78, 64, and 37 are presented to test taker 105 via telephone earpiece 103. Each item is presented at an interval of two seconds immediately followed by the next number and the items presented and the timings of the items presented are stored by the system. After all the items in a list are presented, a delay of four seconds precedes a request to recall the numbers just presented. After each list presentation test taker 105 is asked to enter the numbers recalled into keypad 104 (FIG. 1) or say the numbers into a telephone microphone 106 (FIG. 1a). When two integers are entered into keypad 104 (FIG. 1) or a number is said into microphone 106 (FIG. 1a), the system considers it a response and stores the entry, together with the timings of the response, i.e. amount of time passed since the request to recall the numbers was made. This information includes also the order of the items recalled and any errors made. In FIGS. 1 and 1a, the test taker 105 makes an initial mistake and enters the numbers 79, 64, and 37.

If the caller enters the same number of items that were presented, the next list is automatically presented. If the caller recalls fewer numbers than presented and then waits, the system asks the caller whether they want to go onto the next list. The caller confirms readiness for the next list by pressing a particular number on keypad 104 (FIG. 1) or by saying a particular word into telephone microphone 106 (FIG. 1a).

Fifth, at the conclusion of the list presentations, device 101 computes and/or constructs an analysis of the test results and converts them to voice and presents the analysis to the caller via earpiece 103 and also stores them, together with the identity of the caller on storage device 102. The results are then communicated to a researcher using the test taker as a subject, a medical or psychological provider caring for the test taker, or a researcher using the test taker as a subject, together with an identity code via email, or a web service, or printed out and sent via regular mail.

Separately the test taker logs into a website, presents the paid-for identity number, and enters the mail and email addresses he or she wishes to have the report sent do. The results can also be communicated to the test taker directly by device 101 via the output device 103. The analysis can consist of the raw data (items remembered, order of items remembered and timings of items remembered). It can also include the average number of items remembered and how that number compares with normal subjects or subjects with varying degrees of disease progression (Alzheimer's Disease) or drug usage (marijuana). The analysis can also include the average over the timings recorded in which slow response timings may indicate the presence of Alzheimer's disease or drugs. Slopes of the graphs of response probabilities or response timings of particular items versus item presentation order can also be included in the analysis. These slopes indicate how the test taker's short term memory changes with the order of the item presentation and that may indicate varying degrees of disease progressions or drug usage.

Sixth, the results can be used to obtain permission or a license, e.g., to operate heavy machinery, obtain a driver's license, trade on a bank's trading floor, fly an airplane, go on a spy mission, or perform other relatively risky activities as follows: If the test results are good enough for a particular activity the computational device can respond by issuing a pass code that can be used for activity access. The determination of what is “good enough for a particular activity” is established from past correlations of the memory test results and the particular activity. E.g., if the subject needs to qualify for a driver's license and past experience has indicated that a driver should have a short-term memory that can freely recall three items in order to remember which car in addition to the driver's arrived in which order at a four-way stop sign, then a subject must meet this memory test level to qualify for the short-term memory part of the driver's license qualification.

The efficacy of a neuropharmacological agent on memory can be evaluated with this embodiment. Some subjects would have the agent administered before the test and some would not have it administered so that the effects of the agent on test results can be ascertained. Alternatively the same test takers can also be tested twice, once with a neuropharmacological agent and once without.

To refine the test design, it is convenient to add a human experimenter between the computational device and the telephone speaker in FIG. 1. The human experimenter reads a computer screen that indicates what to say into the telephone. It is also possible to print out the entire test on a sheet of paper so that it can be read into the telephone by the human experimenter. The human experimenter can note common difficulties that the test taker may have. When difficulties arise, the human experimenter is in a better place to fix issues with the test taker than a computer is, thus avoiding the test taker leaving the test with a bad impression.

To perform the above described test, various alternative computational hardware can be used including:
output mechanisms such as a loudspeaker, an electronic paper, a printed paper, a direction to a human presenter, or device connected via a network interface (for example, in a client/server type of configuration)
input mechanisms such as a keyboard, touch screen, a telephone mouthpiece, a microphone, or a other device connected via a network interface (for example, in a client/server type of configuration) to enter the responses with
storage mechanisms including tape drives, hard drives, or other permanent memory devices over a potential long time period.

Any hardware item can be replaced by several items. E.g., one computer can generate an item set and another computer can use that item list and test subjects.

### Second Embodiment

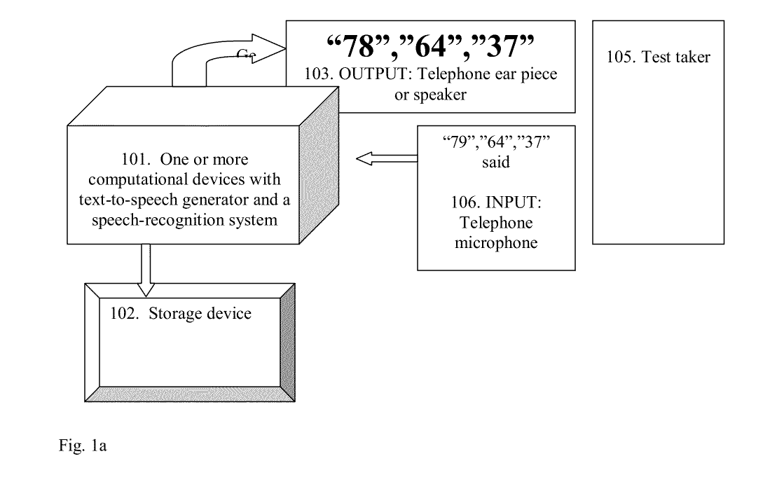

### FIG. 2

The first embodiment can be varied to provide other embodiments and variations.

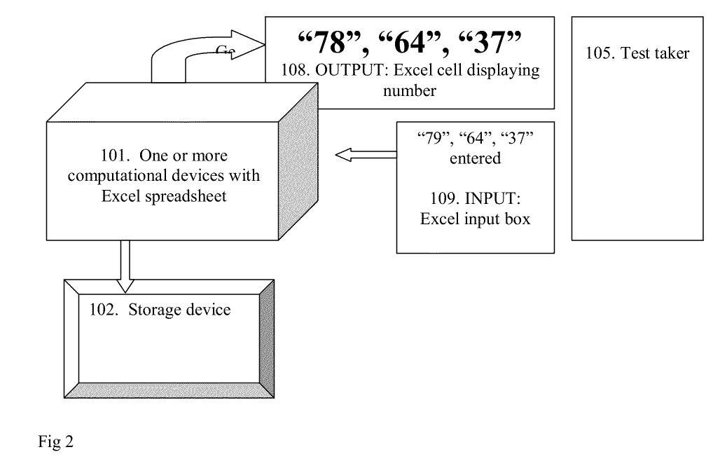

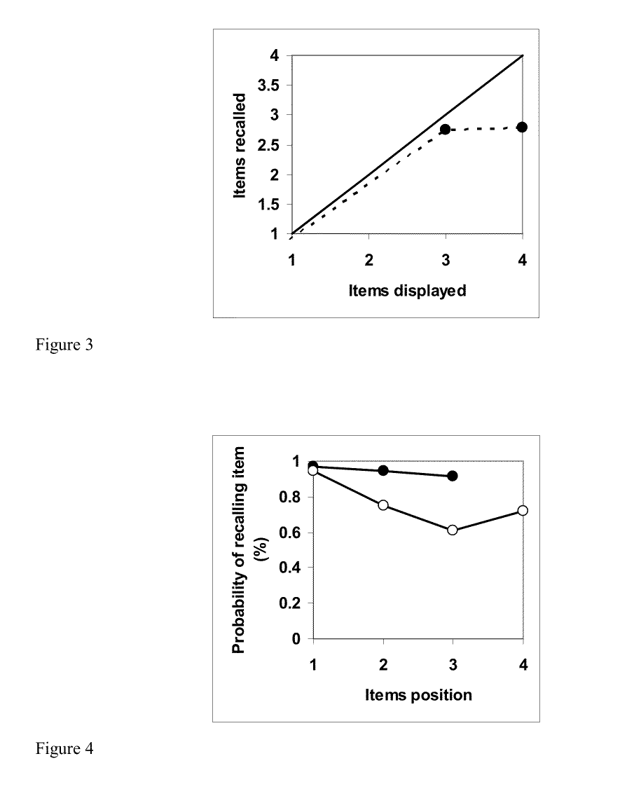

In a second embodiment (FIG. 2), the telephone output device is replaced by a simple cell 108 of a spreadsheet (available in the spreadsheet program sold under the trademark Excel by Microsoft Corp.) and the telephone input device is replaced by an Excel input box 109. The program code for the spreadsheet is appended. Six lists of three two-digit integers were presented, followed by six lists of four two-digit integers. Six test takers of various ages generated the average results shown in FIGS. 3 and 4.

FIG. 3 shows how many items were recalled when three or four items were presented. The broken line is a guide to show what perfect recall would look like. In the figure we see that when three numbers are displayed, on the average 2.7 numbers are recalled. When an additional number is displayed the total recall does not increase. Indeed, for some test takers the total recall decreases. In other words, the disclosed test tests the boundary of short-term memory and answers the question spelled out above: the capacity of short-term memory is three non-chunked items.

When presented with four items, what item does the test takers tend to leave out? The corresponding experimental results are shown in FIG. 4, which depicts the probability of recall of each item as a function of the presentation order. The filled circles are tests with three double-digit integers and the unfilled circles are tests with four double-digit integers. Notice the large discrepancy between the near-perfect result for three numbers and the much worse result for four numbers. For three items the recall is roughly the same for all items. When four items are presented, the recall is as good only for the first item. It is worse for the second item (which is similar to the fourth item) and the worst for the third item.

The disclosed test tends to impose a rehearsal pattern. The test can start out with only two items in a list, and for two items there is only one simple pattern of rehearsal: item1, item2, item1, item2.

The test can then increases the number of items to three. For three items there are two simple rehearsal patterns: item1, item2, item3, item1, item2, item3 and item1, item3, item2, item1, item3, item2.

Because the test taker became used to rehearsing the sequence item1, item 2 when there were two items, they tend to rehearse in the item1, item2, item3 pattern when there are three items.

The evidence includes that for three items test takers will typically recall them in the item presentation order.

When subjects are presented with a list of four items, the reactions to the question “Can you describe what it felt like to go from trying to remember three numbers to four numbers?” were as follows:
“I was disconcerted that the four numbers had started before I expected”
“The numbers are harder to keep in order”
“[It was] A jump”
“It got a bit harder to keep track of things. There was jumbling.”
“I found it much harder. I tried to remember first two items, lost track of them and later ones when the fourth item came along.”
“I found it confusing.”

### Third Embodiment

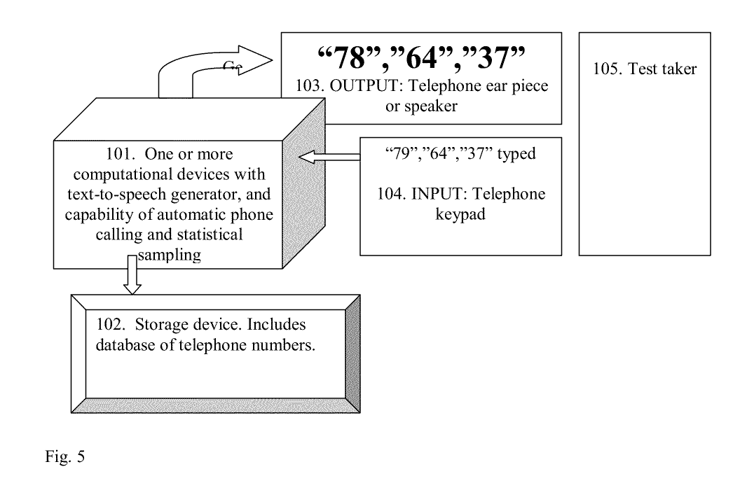

### FIG. 5

In a third embodiment, FIG. 5, computational device 101 initiates a phone call to the test taker (“robocalling”) using a database of phone numbers stored in storage device 102.

This way a whole population can be randomly tested by an epidemiologist (using a statistical sampling by the computational device 101 of the phone numbers in storage device 102) by phone calls much the same way as their votes are polled by political and commercial polling companies. This can be done for a fraction of the cost if each subject had to be called into a hospital setting and tested by a medical worker.

If the database stored in storage device 102 includes not only phone numbers but subscriber age (which is rather easy to obtain from the Internet), the random testing can be limited to subscribers over a particular age to identify common memory issues of aging including Alzheimer's disease. The telephone area codes include location information allowing the testing to probe potential local-specific environmental factors.

The disclosed tests are of low cost and be used to perform massive screenings of Alzheimer's disease.

The disclosed test can also be used for random drug testing of drugs that impair the memory test results, including by the Alcoholics Anonymous or Narcotics Anonymous to keep its members honest about alcohol or drug use.

If the telephone database includes pregnancy status, the effects of pregnancy on short-term memory can be studied. If the telephone database includes potential employees, a company can call them and test their short-term memory.

It can also call those who signed up for a test subscription covering monthly memory tests via robocalls.

A programmer of average skill can implement the robocalling system and interface it with phone number databases.

### Fourth Embodiment

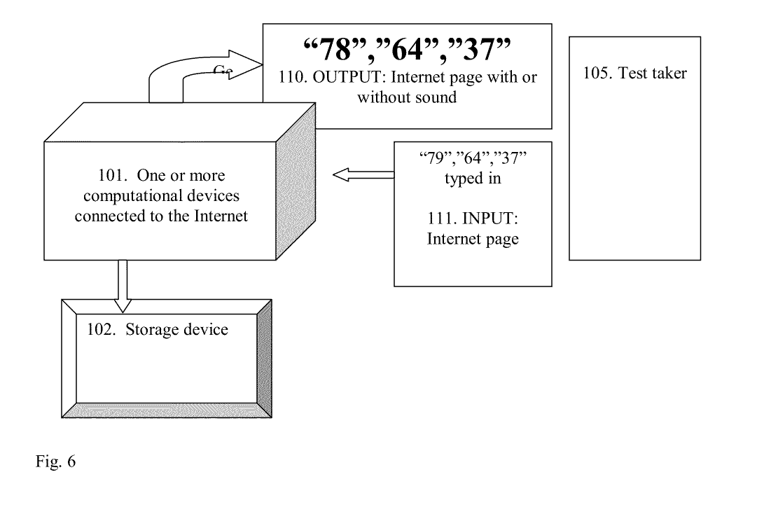

### FIG. 6

In a fourth embodiment, FIG. 6, the test is carried out by a computer via the Internet instead of by a computer via the telephone. This may be useful to younger test takers that prefer interacting with the internet rather than with a telephone. The items can be presented via the screen or via speakers; in the former case it will make the test accessible to people with difficulty hearing.

### Fifth Embodiment

In a fourth embodiment the computational device in FIG. 1 or 1a is a smart phone or other portable computational device. This will make the test easy for the test taker to access, in particular if the test taker wishes to take the test repeatedly with spaced intervals to monitor whether her or his memory is deteriorating or not.

### Sixth Embodiment

In a sixth embodiment, the test in any of the embodiments focuses on the transition from trying to remember three two-digit items to four two-digit items. As the test taker is initially presented with an item beyond the test taker's capacity to recall, the test taker decision can be quick or slow. If the test taker is alert and makes quick decisions he or she can quickly decide on a reasonable strategy. If the test taker makes a slow decision, it will take time away from the rehearsal time and more than the extra item will drop out of memory. The correspondingly lower memory score will reflect this slow decision. Cusack et al (2009, supra) found a similar effect when subjects were asked to remember a visual display of letters and their conclusion was “it is the additional cognitive process that depresses performance at higher set sizes . . . that couples with intelligence, not VWM [visual working memory] per se.”

This decision point in the test probes attention, alertness, sleep deprivation, diseases, syndromes and drugs that affect short-term memory and/or executive decision making.

### Seventh Embodiment

A seventh embodiment uses the previously disclosed embodiments 1-5 to present an analysis that compares individual test results with normal distributions and distributions resulting from impaired memory to ascertain the probability of impaired memory. The analysis can include the previously disclosed Recency-Primacy Shift (RPS; defined above) (Tarnow '011, supra). RPS is relatively independent of a person's age and thus particularly useful to separate out the effects of Alzheimer's disease from normal aging.

The Recency-Primacy Shift is calculated from the item free recall probability versus item presentation order curve by subtracting the normal values from the test taker's values, calculating the slope of the new curve and, to normalize, multiply the slope by the number of items presented. Test takers with Alzheimer's disease have a large non-zero Recency-Primacy Shift (Tarnow '011, supra).

The analysis can also include the timings of the responses and how they compare with those of normal distributions and distributions resulting from impaired memory to ascertain the probability of impaired memory

### Eighth Embodiment

An eighth embodiment stores multiple individual test results over time to track progression of memory impairment or impairment in executive decision making skills.

### Ninth Embodiment

A ninth embodiment adds a particular distraction such as a radio program, a cell phone conversation, lighting changes and scores the corresponding level of distraction by how much the memory test results change.

### CONCLUSIONS, RAMIFICATIONS, SCOPE

According to one or more aspects, my system has one or more of the following advantages:
1. The test taker can take the test without the presence of other people, via telephone or the Internet, minimizing embarrassment and distraction and without the test taker having to use a complex computational device.
2. Using few double-digit integers the test can measure limits of short-term memory straightforwardly without the need for complex mathematics or complex visual test items.
3. This system allows a layperson to see and “feel” their own short-term memory limit without asking a scientist or a computer to analyze the results.
4. The measured limit on short-term memory can be easily probed for memory deficiencies.
5. The test shows how short-term memory free recall limit is three non-chunkable items.
6. A controlled rehearsal process can be constructed and probed for memory deficiencies in terms of the ease with which to locate and internally reactivate test items.
7. The short-term memory overload response can be probed for executive decision deficiencies caused by disease or sleepiness or drug usage.
8. The system allows the construction of a cross-cultural memory test by avoiding language specific word associations and limiting it to integers.
9. The cross-cultural test allows memory deficiencies to be compared and defined across international borders.
10. A high quality test item set can be constructed with low chunkiness that makes the test results less dependent on the particulars of individual associations and a better reflection of the true short-term memory capacity.
11. The test item set can also be constructed to maximize homogeneity which allows high statistical quality with minimal number of list presentations.
12. If the test is one that uses integers, it can be used to test memory across cultures as well as languages.
13. The use of a pass code to indicate that the test taker passed the memory test allows the test result to restrict activities that require a good short-term memory, for example, usage of heavy machinery or driving a car. A bad test result can come from neurological disease, lack of sleep, legal or illegal drug use, all of which can restrict the activities of the test taker with a simple phone call.
14. An unusually good result identifies individuals with special memory abilities; those who can freely recall more than three chunks
15. The test can be used to screen for the presence of diseases or syndromes of short-term memory, including Alzheimer's disease and other neurological conditions. An early diagnosis would make Alzheimer's disease drug trials faster and less expensive for those drugs that claim to prevent Alzheimer's disease.
16. The test can be used to screen for sleepiness and neuropharmacological drug related changes that affect the short-term memory, attention, alertness, and decision making.

The embodiments disclosed overcome one or more of the limitations in prior-art systems described above. They also overcome other limitations that will become apparent upon reading and understanding this patent.

While the foregoing disclosure has been described in some detail for purposes of clarity and understanding, it will be appreciated by one skilled in the art, from a reading of the disclosure, that various changes in form and detail can be made. For example

1. The disclosed integer test can be housed in a small box with a keyboard, display, and computer that can be used for repeated self-testing and self-referencing of one's short-term memory to diagnose Alzheimer's disease, sleepiness, drunkenness, and influences of other neuropharmacological drugs including on impaired drivers.

2. The disclosed integer test can be used for inexpensive mass testing of military recruits to identify persons of exceptional short-term memory ability (good or bad). One common display could be used in conjunction with multiple keyboards.

3. The memory recall test in any of the previous embodiments can be varied to include a recognition test (in which items from the test item set are shown after the initial presentation and the subject is requested to say whether those items were initially presented or not), a cued recognition test (in which items are initially displayed in pairs and the subject is requested to fill in the identity of the paired item when only a single item is displayed), free and cued selective reminding and other memory tests. The responses are different from free recall responses. For example recognition responses are true/false responses and the corresponding timings of the response we need to measure are the times elapsed from the item is presented until the true/false answer is indicated by the test taker. Any of these methods of memory testing is applicable to part or all of the disclosures.

4. The integers in the tests described can be replaced by other item sets, including words and pictures, which probe different parts of the brain than do numbers.

5. There is a lot of data present in the test taker responses that are stored including the order of the response as compared to the order of the presentations (does the test taker respond with the first item first?), the time it takes to recall particular items measured from the original request to respond, etc.

6. The disclosed tests test not only memory but also other brain activity including alertness, sleepiness, executive skills, etc.

7. The disclosed test via phone can be used by researchers to inexpensively follow subject cohorts over time since there is no need for the subjects to meet face-to-face with the researcher to take the memory test.

Thus the full scope of the disclosure should be determined by the appended claims and their legal equivalents.

### COMPUTER PROGRAM LISTING

This code can be pasted into an Excel™ spreadsheet after creating an appropriately named worksheet “Test”.

Sub Macro 1( )
′
′ Macro1 Macro
′ Macro recorded 4/25/2013 by Eugen
′
′ Keyboard Shortcut: Ctrl+q
′
Active Workbook.EnableAutoRecover = False
Dim personname As String
personname = “”
personname = InputBox(“Please enter your name and age separated
by a comma. For example, ‘Eugen, 51’”, “Input Requested”)
If personname = “” Then
MsgBox “No name was provided. The test cannot begin.”
Exit Sub
End If

Set NewSheet = Sheets.Add(Type:=xlWorksheet,

After:=Sheets(Sheets.Count))

NewSheet.Name = personname
Sheets(“Test”).Select

Randomize
MsgBox “You will get 6 lists of 3 and then 6 lists of 4 numbers.
Each list will be followed by a test in which you enter each number
separately in a prompt box. You can enter the numbers in any order.
Try to remember the numbers for the test without using
your voice. The test begins when you click ‘OK’”
Dim numitems As Integer
Dim numtrials As Integer
numtrials = 12 ′has to be EVEN
Dim displayarray(1 To 10) As Integer
Dim correctarray(1 To 10) As Integer
For ii = 1 To numtrials
MsgBox “The next list is ready”
′If ii Mod 2 = 0 Then
′numitems = 4
′Else
′numitems = 3
′End If
If ii > numtrials / 2 Then
numitems = 4
Else
numitems = 3
End If
Dim totalcorrectarray3(1 To 10) As Integer
Dim totalcorrectarray4(1 To 10) As Integer
For i = 1 To numitems
anotherguess:
guess = CInt(100 * Rnd)
If guess < 21 Or guess = 100 Then GoTo anotherguess
If guess Mod 10 = 0 Or guess Mod 11 = 0 Then GoTo anotherguess
If i > 1 Then
If Abs(guess − displayarray(i − 1)) < 10 Then Go To anotherguess
End If
For j = 1 To numitems
If guess = displayarray(j) Then GoTo anotherguess
If Abs(guess − displayarray(j)) Mod 10 = 0 Then GoTo anotherguess
Next
displayarray(i) = guess
Cells(5, 5) = guess
′Application.Wait (1)
Application.Wait Time + TimeSerial(0, 0, 2)

Next
Cells(5, 5) = “”
Application.Wait Time + TimeSerial(0, 0, 2)
′now the person is guessing
Dim dTime As Single
dTime = Timer
Dim answerarray(1 To 10) As Integer
Dim timearray(1 To 10) As Single
For i = 1 To numitems
a = InputBox(“”)
timearray(i) = Timer − dTime
If a = “” Then GoTo done
answerarray(i) = CInt(a)
Next

done:

For i = 1 To 10
For j = 1 To 10
If answerarray(j) = displayarray(i) Then correctarray(i) = 1
Next

nextone:

Next
For i = 1 To numitems
If numitems = 3 Then
totalcorrectarray3(i) = totalcorrectarray3(i) + correctarray(i)
Else
totalcorrectarray4(i) = totalcorrectarray4(i) + correctarray(i)
End If
correctarray(i) = 0
Next
Sheets(personname).Select
For jj = 1 To numitems
Cells(3 * ii, jj) = displayarray(jj)
If Not answerarray(jj) = 0 Then
Cells(3 * ii + 1, jj) = answerarray(jj)
Cells(3 * ii + 2, jj) = timearray(jj)
End If
answerarray(jj) = 0
timearray(jj) = 0
Next

Sheets(“Test”).Select

Next
Sheets(personname).Select
For i = 1 To numitems
Cells(1, i) = totalcorrectarray3(i) / (numtrials / 2)
Cells(2, i) = totalcorrectarray4(i) / (numtrials / 2)
Next
Dim feeling As String

feeling = “”
feeling = InputBox(“Can you describe what it felt like to go from
trying to remember 3 numbers to 4 numbers?”, “Input Requested”)
Cells(1, 15) = feeling
feeling = “”
feeling = InputBox(“Describe any strategy you may have used to
remember the numbers”, “Input Requested”)
Cells(2, 15) = feeling

Sheets(“Test”).Select
Active Workbook.EnableAutoRecover = True
Cells(5, 5) = 0

End Sub

I claim:

1. A method for testing the brain activity of a test taker, comprising:
(a) providing a system selected from the group consisting of a single computational device and multiple computational devices, said system having input, output, and storage mechanisms,
(b) selecting an item set that has a property selected from the group consisting of high homogeneity, low homogeneity, high chunkiness, and low chunkiness,
(c) using said system to store said item set,
(d) presenting a subset of items from said item set to said test taker,
(e) storing said subset of items,
(f) requesting said test taker to respond with a response selected from the group consisting of recalling said subset of items and recognizing said subset of items,
(g) storing said response and timings of said response,
(h) repeating steps (d)-(g) N times where N is selected from the group consisting of no times, one time, two times, and several times,
whereby said test taker is tested with an item set with said property.

2. The method of claim 1, further including administering a neuropharmacological agent to said test taker prior to step (d), whereby the effect of said neuropharmacological agent on said response can be ascertained.

3. The method of claim 1, further including using said system to request a language preference from said test taker, receive said language preference from said test taker, and
present said subset of items in the requested language.

4. The method of claim 1, further including using said system to construct an analysis selected from the group of restating said response and said timings of said response, calculating averages of said response and said timings of said response, calculating a Recency-Primacy Shift of said response, calculating a slope of a graph of said response versus order of item presentation, identifying whether said response indicates said test taker may have Alzheimer's disease, identifying whether averages of said response and said timings of said response indicates said test taker may have Alzheimer's disease, identifying whether a Recency-Primacy Shift of said response indicates said test taker may have Alzheimer's disease, identifying whether a slope of a graph of said response versus order of item presentation indicates said test taker may have Alzheimer's disease.

5. The method of claim 1, further including using said system to issue a pass code if said response indicates a particular activity could be effectively carried out.

6. The method of claim 1 wherein said item set consists of two digit integers excluding two digit integers selected from the group consisting of no integers, integers that are multiples of 10, integers that are multiples of 11, integers that are related by transposition to already presented integers, integers that are one number away from integers already presented and integers that are less than three numbers away from integers already presented.

7. An inexpensive method for testing brain activity of a test taker selected from the group consisting of one human subject and multiple human subjects comprising:
(a) providing a system selected from the group consisting of a single computational device and multiple computational devices, said system having input, output, phone calling and storage mechanisms,
(b) using said system to store an item set,
(c) initiating a phone call by a first entity selected from the group consisting of said system, said test taker, a researcher using said test taker as a subject, a medical provider caring for said test taker, and a psychological provider caring for said test taker,
(d) using said system to present a subset of items from said item set via said phone call to said test taker,
(e) requesting said test taker to respond with a response selected from the group consisting of recalling said subset of items as having been just presented and recognizing said subset of items as having been just presented,
(f) storing said response and timings of said response,
(g) repeating step (d)-(f) N times where N is selected from the group consisting of no times, one time, two times, and several times,
whereby said response and said timings of said response by said test taker are stored via said phone call.

8. The method of claim 7, further including using said system to construct an analysis selected from the group of restating said response and said timings of said response, calculating averages of said response and said timings of said response, calculating a Recency-Primacy Shift of said response, calculating a slope of a graph of said response versus order of item presentation, identifying whether said response indicates said test taker may have Alzheimer's disease, identifying whether averages of said response and said timings of said response indicates said test taker may have Alzheimer's disease, identifying whether a Recency-Primacy Shift of said response indicates said test taker may have Alzheimer's disease, identifying whether a slope of a graph of said response versus order of item presentation indicates said test taker may have Alzheimer's disease.

9. The method of claim 8 further including presenting said analysis to a party selected from the group consisting of said test taker, a researcher using said test taker as a subject, a medical provider caring for said test taker, and a psychological provider caring for said test taker.

10. The method of claim 7, further including administering a neuropharmacological agent to said test taker prior to step (d), whereby the effect of said neuropharmacological agent on said response can be ascertained.

11. The method of claim 7, further including using said system to request a language preference from said test taker, receive said language preference from said test taker, and present said subset of items in the requested language.

12. The method of claim 7, further including using said system to issue a pass code if said response indicates a particular activity could be effectively carried out.

13. The method of claim 7 wherein said item set consists of two digit integers excluding two digit integers selected from the group consisting of no integers, integers that are multiples of 10, integers that are multiples of 11, integers that are related by transposition to already presented integers, integers that are one number away from integers already presented and integers that are less than three numbers away from integers already presented.

14. A method for testing brain activity of a test taker, comprising:
(a) providing a system selected from the group consisting of a single computational device and multiple computational devices, said system having input, output, and storage mechanisms for storing an item set,
(b) using said system to present N items from said item set to said test taker,
(c) storing said N items,
(d) requesting said test taker to respond with a response selected from the group consisting of recalling said N items and recognizing said N items,
(e) storing said response and timings of said response,
(f) repeating steps (b)-(e) W times where W is selected from the group consisting of no times, one time, two times, and several times,
(g) repeating steps (b)-(f) with N=N+1,
whereby the properties of said response are stored for N items and for N+1 items.

15. The method of claim 14, further including administering a neuropharmacological agent to said test taker prior to step (b), whereby the effect of said neuropharmacological agent on said response can be ascertained.

16. The method of claim 14, further including using said system to request a language preference from said test taker, receive said language preference from said test taker, and present said subset of items in the requested language.

17. The method of claim 14, further including using said system to construct an analysis selected from the group of restating said response and said timings of said response, calculating averages of said response and said timings of said response, calculating a Recency-Primacy Shift of said response, calculating a slope of a graph of said response versus order of item presentation, identifying whether said response indicates said test taker may have Alzheimer's disease, identifying whether averages of said response and said timings of said response indicates said test taker may have Alzheimer's disease, identifying whether a Recency-Primacy Shift of said response indicates said test taker may have Alzheimer's disease, identifying whether a slope of a graph of said response versus order of item presentation indicates said test taker may have Alzheimer's disease.

18. The method of claim 14, further including using said system to issue a pass code if said response indicates a particular activity could be effectively carried out.

19. The method of claim 14 wherein said item set consists of two digit integers excluding two digit integers selected from the group consisting of no integers, integers that are multiples of 10, integers that are multiples of 11, integers that are related by transposition to already presented integers, integers that are one number away from integers already presented and integers that are less than three numbers away from integers already presented.

20. A method for more efficiently testing brain activity of a test taker by using a non-trivial dynamical set change rule comprising:
(a) providing a system selected from the group consisting of a single computational device and multiple computational devices, said system having input, output, and storage mechanisms for storing an item set,
(b) presenting an item from said item set to said test taker,
(c) changing said item set using a non-trivial dynamical set change rule selected from the group consisting of remove items, substitute items, and add items,
whereby said item set is changed to obtain a preferred property selected from the group consisting of increased homogeneity, decreased homogeneity, increased chunkiness and decreased chunkiness.

21. The method of claim 20, further including using said system to request a language preference from said test taker, receive said language preference from said test taker, and present said subset of items in the requested language.

22. The method of claim 20 wherein said item set consists of two digit integers excluding two digit integers selected from the group consisting of no integers, integers that are multiples of 10, integers that are multiples of 11, integers that are related by transposition to already presented integers, integers that are one number away from integers already presented and integers that are less than three numbers away from integers already presented.

23. A method for systematically improving a test item set and a set of dynamical set change rules for testing the brain activity of a test taker selected from the group consisting of one human subject and multiple human subjects comprising:
(a) providing a system selected from the group consisting of a single computational device and multiple computational devices, said system having input, output, and storage mechanisms,
(b) using said system for storing a first item set and a first set of dynamical set change rules,
(c) presenting a subset of items from said first item set to said test taker using said first set of dynamical set change rules,
(d) storing said subset of items from said first item set,
(e) requesting said test taker to respond with a first response selected from the group consisting of recalling said subset of items and recognizing said subset of items,
(f) storing said first response and timings of said first response,
(g) repeating steps (c)-(f) X times where X is selected from the group consisting of no times, one time, two times, and several times,
(h) constructing a second item set by varying said first item set by a method selected from the group consisting of substituting items, adding items, and deleting items,
(i) constructing a second set of dynamical set change rules by varying said first set of dynamical set change rules by a method selected from the group consisting of substituting dynamical set change rules, adding dynamical set change rules, and deleting dynamical set change rules,
(j) storing said second item set and said second set of dynamical set change rules,
(k) presenting a subset of items from said second item set to said test taker using said second set of dynamical set change rules,
(l) storing said subset of items from said second item set,
(m) requesting said test taker to respond with a second response selected from the group consisting of recalling said subset of items from said second item set and recognizing said subset of items from said second item set,
(n) storing said second response and timings of said second response,
(o) repeating steps (k)-(n) Y times where Y is selected from the group consisting of no times, one time, two times, and several times,
(p) selecting from the combination of said first item set and said first set of dynamical set change rules and the combination of said second item set and said second set of dynamical set change rules a preferred combination of an item set and a set of dynamical set change rules with a preferred property selected from the group consisting of increased homogeneity, decreased homogeneity, increased chunkiness and decreased chunkiness,
(q) substituting said preferred item set and said preferred set of dynamical set change rules for said first item set and said first set of dynamical set change rules,
(r) repeating steps (b)-(q) Z times where Z is selected from the group consisting of no times, one time, two times, and several times,
whereby said first item set and said first set of dynamical set change rules obtains said preferred property.

24. The method of claim 23 wherein said first item set initially consists of two digit integers and said first set of dynamical set change rules consists of the empty set.
